(19) United States
(12) Patent Application Publication
(10) Pub. No.: US 2017/0350898 A1
(43) Pub. Date: Dec. 7, 2017

(54) METHOD AND APPARATUS FOR DETECTING VECTOR-BORNE DISEASES IN HUMANS

(71) Applicant: VETERINARY DIAGNOSTICS INSTITUTE, INC., Simi Valley, CA (US)

(72) Inventor: RANDY RINGOLD, West Hills, CA (US)

(73) Assignee: VETERINARY DIAGNOSTICS INSTITUTE, INC., Simi Valley, CA (US)

(21) Appl. No.: 15/524,078

(22) PCT Filed: Nov. 5, 2015
(86) PCT No.: PCT/US15/59333

§ 371 Date: May 3, 2017

Related U.S. Application Data
(60) Provisional application No. 62/076,474, filed on Nov. 6, 2014.

(51) Int. Cl. G01N 33/574; C12Q 1/04; G01N 33/569

(52) U.S. Cl. CPC G01N 33/57484; C12Q 1/04; G01N 33/569

(57) ABSTRACT

Screening human subjects for vector-borne disease is provided and carried out using thymidine kinase 1 alone or in combination with c-reactive protein as biomarkers which are measured in a blood sample, and the measurements are used to compute an index that allows a practitioner to compare results from different subjects and between different populations of subjects, and the screening for vector-borne disease can also be used on subjects that are undergoing treatment to monitor the progress of an infection and the results of the treatment.

### CROSS-REFERENCE TO RELATED APPLICATION

The present application claims priority to international patent application No. PCT/US15/59333, filed on Nov. 5, 2015, which claims priority to U.S. provisional patent application No. 62/076,474 filed on Nov. 6, 2014, the content of each of which in its entirety is included herein by reference.

### FIELD OF THE INVENTION

The invention relates to a method and apparatus for detecting vector-borne diseases. More specifically, the invention comprises a method and apparatus for diagnosing the presence of vector borne diseases in human subjects using the measurement of one or more biomarkers.

### BACKGROUND OF THE INVENTION

Vector-borne diseases (VBD) is a category of disease where an infectious micro-organism (a pathogen) is generally carried by a vector and transmitted to other bodies through the vector's natural behavior such as blood-sucking activity. Arthropods are the vectors for many disease-causing micro-organisms which are inoculated into a victim's body by sting and/or feeding on the victim's body. The most common arthropods that serve as vectors, in the case of humans and house pets or farm animals include blood sucking insects, such as mosquitoes, fleas, lice and other biting insects, and blood sucking arachnids, such as mites and ticks.

Typically, vectors become infected by a disease-causing microbe while feeding on infected vertebrates (e.g., birds, rodents, other larger animals, or humans). The microbe is then transmitted to other animals. In almost all cases, an infectious microbe must infect and multiply inside the arthropod before the arthropod is able to transmit the microbe, e.g., through its salivary glands.

Vector-borne diseases represent a varied and complex group of diseases, some of which are known and yet new syndromes that are still being uncovered. Borreliosis (Lyme disease) is the most frequently reported vector borne infection in the United States. Other known diseases include for example anaplasmosis, babesiosis, dirofilariosis, ehrlichiosis, leishmaniosis, rickettsiosis and thelaziosis.

Many of the vector-borne diseases can cause serious (or even life-threatening) clinical conditions. Concerning dogs and cats, a number of these diseases carried by the latter two species may have zoonotic potential, i.e. potentially transmitted to humans (see table 1).

TABLE 1

| Disease | Vector |
|---|---|
| Leishmaniosis | Sand fly |
| Borreliosis | Tick |
| Bartonellosis | Flea, Tick |
| Ehrlichiosis | Tick |
| Rickettsiosis | Tick, Flea |
| Anaplasmosis | Tick |
| Dilofilariosis | Mosquito |
| Yersiniosis | Flea |
| Tularaemia | Tick |
| Coxiellosis | Tick |
| Tick-borne encephalitis | Tick |
| Louping ill | Tick |
| West Nile virus encephalitis | Mosquito |
| Trypanosomiosis | Triatoma bugs |

The incidence of vector-borne diseases in both humans and animals is increasing. Today, vector-borne diseases pose a growing global threat as they continue their spread far from their traditional geographical and temporal restraints as a result of changes in both climatic conditions and humans and pets travel patterns, exposing new populations to previously unknown infectious agents and posing unprecedented challenges to the medical community and veterinarians.

Without treatment, Vector-borne diseases are often characterized by three stages: 1) acute phase, 2) sub-clinical phase and 3) chronic phase. In humans, for example, the acute phase begins within 8-20 days following transmission and lasts for several weeks, and may be manifested by fever, depression, and weight loss. The subclinical phase may last from several months to years in which the host remains persistently infected without showing clinical signs. The last stage, chronic phase, resembles the first phase, but hemorrhaging or edema, and in severe cases death, may occur.

Vector-borne pathogens typically infect portions of the hematopoietic system, such as red blood cells, T-cell, monocytes, or granulocytes. The pathogen uses the host cell to replicate. The pathogen may remain within the hematopoietic system or transmit through the bloodstream to invade other cell lines within specific organs, such as the liver.

Vector-borne pathogens have evolved unique mechanisms to persist and/or multiply within a host. Pathogens may have lost cell membrane Lipopolysaccharide (LPS) and peptidoglycan, which would otherwise activate the host's innate immune defense mechanisms. Pathogens may manipulate the vector's target neutrophil, which is otherwise designed to destroy the pathogen or prevent the establishment of infection in a rather benign erythrocyte. Pathogens may suppress innate and adaptive immune responses to favor pathogen's survival, and/or express extensive antigenic variation in immunodominant surface proteins to permit evasion of the immune response.

If treated, vector-borne diseases are generally responsive to antibiotic therapy, although, in some patients symptoms may continue for months after treatment. In addition, early detection of VBD plays a crucial role in the success of the treatment and the prevention of the spread of the disease. However, the diagnosis of vector-borne pathogens may be challenging, because clinical signs are frequently non-specific, and serological assays designed to detect the presence of antibodies to the pathogen frequently yield false negative results due to the immunosuppressive capability of the pathogen that prevents the production of antibodies. False positives also result in patients that have previously had a vector infection and still retain antibodies to the pathogen.

Since VBD pathogens such as B. burgdorferi (Lyme disease) are very difficult to culture and post-treatment patients will remain serologically positive, there is currently no method to determine if the infection has truly been eradicated. The use of direct antigen detection with polymerase chain reaction (PCR) has a high degree of accuracy due to the direct detection of the pathogen however it may also have false-negatives due to the transient presence of the pathogen in the blood stream at the time of sampling.

Because of the challenge to diagnose VBD and monitor the health status of treated patients, the cost can be prohibitively expensive. For example, the Centers for Disease Control (CDC) have estimated that approximately 10% to 20% of individuals may experience Post-Treatment Lyme Disease Syndrome (PTLDS)—a set of symptoms including fatigue, musculoskeletal pain, and neurocognitive complaints that persist after initial antibiotic treatment of Lyme disease. The latter symptoms are similar to those experienced by patients with Muscular Dystrophy (MS). As a result, MS patients are routinely evaluated for VBD. On the other hand, those previously infected retain a positive serological titer, which makes ruling out of a vector borne infection challenging.

It is estimated that approximately 10-20% of Lyme disease treated patients will have PTLDS and the CDC estimates this number may be low due to lack of a sensitive biomarker. As a result, these patients require considerably more diagnostic procedures and healthcare services. In the United States, studies have shown PTLDS patients consume approximately $4,000 more in healthcare services than matched controls.

Therefore, there is a need for cost effective and least invasive screening methods that identify subjects with a VBD. Those patients that are screened as positive may undergo further diagnostic workup to identify the infecting pathogen and devise appropriate treatment. A method and system to determine therapeutic response and/or relapse would provide healthcare professionals valuable information on their patient's status so further treatment and patient care management can be conducted quickly and cost-effectively.

### SUMMARY OF THE INVENTION

Currently, there is a lack of methods and systems for cost effectively screening for subjects that have a high probability of being affected by a vector-borne disease. Moreover, for monitoring the health status of a subject having been treated (or under treatment) for vector-borne disease, the current approaches are, in addition of being expensive, yield demonstrably a high percentage of false positives.

The invention provides a method and system that enable a practitioner to screen for vector-borne disease in human subjects using one or more biomarkers. Whether symptoms indicative of a disease are (or are not) already displayed by the subject, an implementation of the invention enables the practitioner to reveal the presence of a vector-borne disease, which may lead to further diagnoses.

The invention utilizes Thymidine kinase type 1 alone or combination with C-Reactive Protein (CRP) in several methods for enabling a practitioner to screen for VBD.

In a first method (Method 1), the invention provides a method of computing a vector-borne disease index (VBI1) using the biomarkers: TK1 and CRP. The measured values of TK1 and CRP, in a blood sample, are discretized according to a mapping disclosed in the invention. A product of the discrete value of each biomarker and a corresponding coefficient is calculated. The VBI1 index is obtained by summing the products obtained from all biomarkers. The discretization maps and the coefficients are optimally set such that an index value greater that one (1) is indicative of a high probability of the presence of VBD and should be considered for further diagnoses.

A second method (Method 2), also utilizes the biomarkers TK1 and CRP to compute a an index (VBI2), and is comparatively simpler to compute than Method 1. The latter index is computed using the amount of biomarker directly measured in a blood sample. The index takes the values of “0” or “1”, based on a mapping provided by the invention.

A third method (Method 3), utilizes biomarker TK1 only and provides a fast screening a practitioner may use when the invention is being used with other type of screenings. For example, if a subject has already been ruled out as being affected by Lymphosarcoma (LSA) and Irritable Bowel Disease (IBD), then Method 3 of the invention may be used as a fast screening for VBD.

Prior art methods for screening for pathogen-caused disease rely on detecting anti-pathogen antibodies to indicate an ongoing infection. The latter methods yield false positives if the subject still carries the antibodies even after the infection is finished. Because the biomarkers used in the invention are indicative of the health status of the subject, the invention is suitable for monitoring the infection status is a subject that may have been treated for vector-borne disease.

### BRIEF DESCRIPTION OF THE DRAWINGS

FIG. 1 is a flowchart diagram representing steps involved in developing a method for detecting and/or differentiating the presence of vector-borne diseases, in accordance with an embodiment of the invention.

FIG. 2 is a bar chart representing statistics of thymidine kinase 1 activity level in several groups affected, respectively, by irritable bowel disease, lymphosarcoma and Lyme disease, and a normal (unaffected) group.

FIG. 3 is a bar chart representing statistics of c-reactive protein concentration for several groups affected, respectively, by irritable bowel disease, lymphosarcoma and Lyme disease, and a normal (unaffected) group.

FIG. 4 plots the results of the Receiver Operating Characteristic (ROC) analysis carried out using TK1, CRP, VBI1 and VBI2.

FIG. 5 plots the results of the Receiver Operating Characteristic (ROC) analysis carried out with TK1 data alone, CRP data alone, VBI1 and VBI2 in a sample of subjects that excludes those subjects with Lymphosarcoma and Irritable Bowel Disease.

FIG. 6 plots the computed values for sensitivity and the specificity as a function of TK cut-off value for a sample of subjects comprising IBD, LSA, Lyme and Normal subjects.

FIG. 7 plots the computed values for sensitivity and the specificity as a function of CRP cut-off values for a sample of subjects comprising IBD, LSA, Lyme and Normal subjects.

FIG. 8 plots the computed values for sensitivity and the specificity as a function of cut-off values of the vector-borne index according to method 1 (VBI1) for a sample of subjects comprising IBD, LSA, Lyme and Normal subjects.

FIG. 9 plots the computed values for sensitivity and the specificity as a function of cut-off values of the vector-borne index according to method 2 (VBI2) for a sample of subjects comprising IBD, LSA, Lyme and Normal subjects.

### DETAILED DESCRIPTION

The invention provides a method and system that enable a practitioner to screen for a vector-borne disease in human or other mammalian subjects using one or more biomarkers. Whether symptoms indicative of a disease are (or are not) already displayed by the subject, an implementation of the invention enables the practitioner to reveal the presence of a vector-disease, which may lead to further diagnoses.

The invention utilizes any number of biomarkers that are indicative of dysregulated proliferation, such as Thymidine kinase type1. Furthermore, the invention may utilize any of the acute-phase proteins (APPs) as a biomarker. The increase or the decrease in the concentration of any number of APPs may be used to establish the suppression of an inflammatory response by a vector-borne pathogen.

In humans, the invention provides a screening method for vector-borne diseases using thymidine kinase type 1 (TK1) alone or in conjunction with c-reactive protein (CRP). The invention provides a method of computing a vector-borne disease index, which enables different practitioners to compare the results of tests, according to the invention, from one subject to another, and the results from two or more different testing institutions. The latter index is obtained by first computing the product of the measurement of each biomarker and a corresponding weighting coefficient, the product is then digitized according to a discretization map, then the vector-borne disease index (VBI) is computed by summing the discretization value over all biomarkers. The invention provides discretization maps that are optimally set such that an index value greater that one (1) is indicative of a high probability of the presence of VBD and should be considered for further diagnoses.

The invention provides in particular a method and apparatus, a screening tool, by which a practitioner determines whether a human may be affected by a vector-borne disease (VBD), as in contrast with other ailments that may (or may not) display similar visible symptomatic signs as VBD. The invention also provides a monitoring tool which a practitioner may use to follow the progress of subjects undergoing treatment for VBD.

In the following description, numerous specific details are set forth to provide a more thorough description of the invention. It will be apparent, however, to one skilled in the pertinent art, that the invention may be practiced without these specific details. In other instances, well known features have not been described in detail so as not to obscure the invention. The claims following this description are what define the metes and bounds of the invention.

The present disclosure shares some aspects of the concepts and the methods described in International patent applications No. PCT/US12/23135 and number PCT/US14/64453, and U.S. application Ser. No. 13/672,677, now U.S. Pat. No. 8,940,869, U.S. application Ser. No. 13/672,687 now U.S. Pat. No. 8,969,022, and U.S. patent application Ser. No. 14/372,328. The content of each of the latter references is included in its entirety in the present disclosure by reference.

### Terminology

Abbreviations “TK” and TK1, as used in the disclosure, interchangeably refer to thymidine kinase type 1. Thymidine kinase as a biomarker may be measured using its enzymatic activity as a marker for its presence, for example, in the blood. The activity level is usually provided as Unit per volume of blood. The scope of the invention encompasses however all available means for determining the amount of TK1 in the blood.

Throughout the description, the terms individual, subject or patient may refer to an animal subject or a person whose biological data are used to develop and/or use an implementation of the invention. The subject may be normal (or disease-free) or showing any combination (e.g., including absence) of symptoms.

The term biomarker refers to any indicator of the health condition of a subject, and may be collected and/or the presence of which measured through any of its manifestations such as enzymatic activity, mass, concentration, cell count, cell shrinkage/shape, deoxyribonucleic acid (DNA) and/or ribonucleic acid (RNA) genetic level of expression or any aspect of the biochemical or the physiological markers that may be related to one or more health conditions. Furthermore, one or more markers maybe collected from one or more body parts (e.g., bodily fluid or tissue). Moreover, for the purpose of designing health status indices (see below) a biomarker data may be any related data that may be considered for diagnosing a disease (or the probability of occurrence thereof) such as age, sex, any biometric data, genetic history (e.g., parent's health status or presence of any affection in the family) or any other data that may contribute to the diagnosis of a disease.

In the disclosure the measurement of biomarkers are typically concerned with measuring the concentration (or the activity level) of the biomarker in the blood serum. One with ordinary skills in the pertinent art would recognize that the invention may be practiced using other body fluids such as cerebrospinal fluid, lymph or any other body fluid for which the invention has been implemented. In addition, implementations of the invention may adequately select more than one body fluid for testing for each or any number of biomarkers considered in a test of detecting VBD.

The term “index” is used throughout the disclosure to refer to a dependent variable that is calculated using two or more data inputs such as the level of a biomarker in the blood stream. An index is computed with the goal of classifying subjects into groups based on disease status. For example, a subject that may be apparently healthy (e.g., showing no signs of VBD), but that has been diagnosed with VBD, would have an index value that reflects the health status, in accordance with embodiments of the invention.

The term “user” may be used to refer to a person, machine or a computer program acting as or on behalf of a person.

In using an enzyme as a biomarker, the level of activity of the enzyme may depend on the type of substrate in the test kit, in addition to other parameters such as temperature and pH. Thus, the disclosure considers any adjustments to the calculation/measurement of the enzymatic activity a practitioner may make to practice the invention as inherent steps required for specific implementations of the invention without deviating from the concept of the invention.

### Diagnosing Vector-Borne Disease

The invention aims at providing cost-effective easy to implement screening for VBD. Therefore, an implementation for screening for VBD in accordance with the invention requires basic laboratory equipment for measuring proteins and/or enzymatic activity levels in body fluids, comprising body fluid collection kits (e.g., red top tubes, needles and syringes), body fluid storage and handling equipment, blood serum separation tools (e.g., centrifuges), test tubes and any other machine or tools for a laboratory test. The invention may be practiced using any available test kits for measuring any target biomarker for a specific implementation.

An embodiment of the invention may be an apparatus, system, kit or any product implementation that enables a person with ordinary skills in the medical or veterinary fields to carry out the steps of the invention. In addition to laboratory equipments for collecting blood samples, extracting biomarkers and measuring the biomarkers, embodiments of the invention comprise computation means such as electronic computers, software program product and any product that may involved in providing a product for screening for VBD in accordance with the invention.

Inflammation is a process triggered in living bodies (e.g., in response to an infection) to defend it against foreign invasion by activating a cascading sequence of events including the formation of antibodies. Vector-borne pathogens have evolved to suppress this inflammatory host response to the infection by the pathogen.

An inflammatory process leads to the activation of the cytokine network. In the early phase of this process, proinflammatory cytokines (TNF-α, IL-1β, INF-γ and IL-12) are released. The activity of proinflammatory cytokines is counteracted by the production of anti-inflammatory cytokines (IL-4, IL-10, IL-13 and TGF-β) and soluble inhibitors of proinflammatory cytokines (soluble TNF-α receptor, soluble IL-1 receptor, and IL-1 receptor antagonist).

In response to the formation of cytokines, a complex series of reactions are initiated called the acute-phase response (APR). These reactions aim to prevent ongoing tissue damage, isolate and destroy the infectious organism (if present) and activate the repair processes necessary to restore the host/organism's normal function. The acute-phase response is characterized by leukocytosis, fever, alterations in the metabolism of many organs as well as changes of the concentration of various acute-phase proteins (APPs) in the blood plasma.

Acute-phase proteins (APPs) have been defined as any protein the concentration of which in the plasma changes by at least twenty five percent (25%) during an inflammatory disorder. Those proteins the concentration of which increases are defined as positive acute-phase proteins (e.g., fibrinogen, serum amyloid A, albumin, C-reactive protein), and those proteins the concentration of which decreases are defined as negative acute-phase proteins (e.g., albumin, transferrin, insulin growth factor I).

For example, C-reactive protein (CRP) is a major APP and has been shown to be an effective measure of general inflammation. The concentration of CRP or any serum APP level correlates to both the severity and the duration of the inflammatory stimuli.

The invention utilizes any of the acute-phase proteins as a bio-marker. The increase or the decrease in the concentration of any number of APPs may be used to establish the suppression of an inflammation by a vector-borne pathogen. Furthermore, the invention may utilize any number of biomarkers that are indicative of dysregulated proliferation, such as Thymidine kinase type1.

Thymidine kinase type 1 (TK1) is a salvage enzyme involved in the synthesis of DNA precursors. Thymidine kinase is expressed only in phase S though G2 of cell division (Mitosis). TK1 levels have been shown in numerous studies, both in humans and animals, to correlate with the proliferative activity of dysregulated replication, a hallmark of tumor disease. Serum TK1 concentrations have been studied in human and veterinary applications.

The study upon which the invention is based hypothesizes that TK1 may be elevated in situations where non-neoplastic dysregulated cellular division occurs leading to a false positive result. This may happen when a pathogen invades a host cell and uses cellular processes in the replication of the pathogen. As shown below, human subjects infected by vector-borne pathogens have an increased TK1 concentration, presumably due to the pathogen's replication.

Embodiments of the invention may utilize the measure of TK1 activity in combination with measuring the concentration of one or more APPs, in order to evaluate the probability that a mammal is a carrier of VBD.

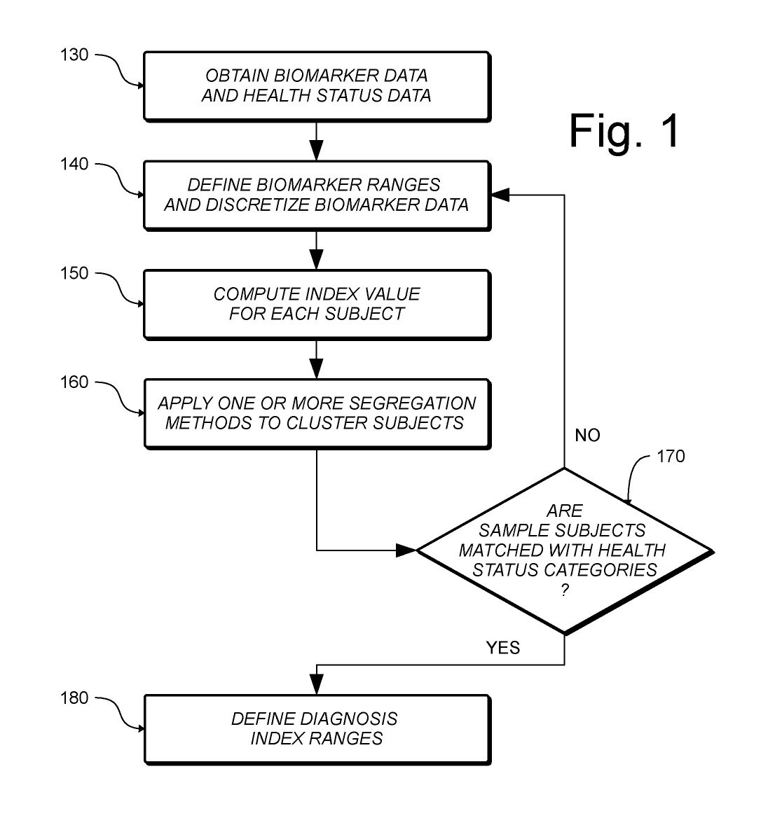

FIG. 1 is a flowchart diagram representing steps involved in developing a method for detecting and/or differentiating the presence of vector-borne diseases, in accordance with an embodiment of the invention.

Step 130 represents collecting data from a group of subjects. The group of subjects may be a sample of subjects comprising normal subjects (i.e. healthy) or unaffected by VBD, and affected subjects showing any level of severity of symptoms and/or other indicators. Bodily fluids, tissue or any other body sample may be appropriately collected in order to measure the level of each biomarker of a set of biomarkers, such as Thymidine kinase, C-reactive protein etc.

In addition, the subjects may undergo a plurality of tests, such as histological, radiological tests or any other test designed to establish the presence or absence of the target disease(s). Other tests may be conducted on each subject to either further confirm VBD or rule out other diseases that may share common symptoms with VBD.

Moreover, other non-disease related data may also be considered. The latter data comprise age, sex, any biometric data, genetic history (e.g., parent's health status or presence of any affection in the family) or any other data that may contribute to the diagnosis of a disease.

The level of each biomarker may be expressed in one or more unit types that characterizes the level of the presence of the biomarker in the body fluid/tissue under consideration. Thus, an enzyme may be characterized by the level of its enzymatic activity, a protein, a hormone or any other biomarker may be expressed by a concentration level such as its mass or moles per volume of tissue or bodily fluid.

Step 140 represents the process of defining range values for each biomarker, and involves discretizing the data, which comprises attributing a score number to each previously defined range of a biomarker level. For example the level of thymidine kinase may be represented by three ranges, the first range may be attributed the value zero (0), the second range may be attributed the value one (1) and the third range may be attributed the value two (2).

Step 150 represents computing an index value for each subject as follows:

$$I = {\sum }_{i = 1}^{i = N} {C}_{i} \cdot {L}_{i} \qquad ( 1 )$$

where the index value “I” for each subject may be the sum of the product of the score level “L” (e.g., computed at step 140) and a coefficient “C” associated with the “i^{th}” data input for a number “N” of data inputs (e.g., biomarker level, age, biometric data etc.). The coefficient “C” may be determined empirically as shown below at steps 160 and 170.

Step 160 represents applying one or more methods for segregating subjects using the health status data and the computed index values. For example, the method of segregation may be the Receiver Operating Characteristic (ROC) curve analysis. ROC curve analysis is a well known method in the medical field for determining whether a correlation between the level of a biomarker may serve as an indicator of the presence of a health condition. The latter is possible for example when there is a strong correlation between the amount of a substance in the body (e.g., high cholesterol) and a health condition (e.g., sclerosis of blood vessels).

Using the ROC curve analysis on the index values of all subjects in the group, it is possible to determine whether there is a cutoff value capable of classifying individuals into groups matching their health status. For example, if subjects carrying a disease are labeled as positive and the non-carriers are labeled as negative, the ROC curve analysis may yield a threshold that classifies the subjects into an above and a below-threshold groups matching the health statuses carrier and non-carrier of the disease, respectively. There may be false positives and false negatives for each chosen cutoff value in the range of possible values. The rate of success in determining true positive cases is called “Sensitivity”, whereas the rate of success in determining true negative cases is called “Specificity”. Sensitivity and specificity for a plurality of cutoff values are computed. Sensitivity and Specificity are rates, and thus may be expressed in the range of zero (0) to one (1), or as a percentage from zero (0) to one hundred percent (100%). The results are plotted as Sensitivity values versus one (1) (or 100% depending on the unit of choice) minus the corresponding specificity. The area under the curve (AUC) reveals whether ROC analysis may be a valid classifier of the data: the closer the AUC is to 100%, the better classifier is the ROC analysis. On the contrary, the ROC analysis may not be considered for classification purposes if the AUC is closer to 50%, which is considered close to a random process. In general, the ROC method of analysis may be considered valid, if the AUC is at least 0.8 (i.e. 80% of the total possible area under the curve).

Moreover, each threshold value yields a “Sensitivity” and “Specificity”. In populations where ROC analysis appears adequate, the “Sensitivity” curve decreases as the “Specificity” increases. At a particular threshold, the apex, the total of Sensitivity and Specificity is at a maximum. The apex is typically chosen as the threshold of classification if it yields a Sensitivity and Specificity each above 0.85, otherwise a threshold for Specificity and a threshold for Sensitivity may be respectively selected to yield a success rate of at least 0.85.

ROC analysis is one of any existing methods that may be utilized in embodiments of the invention to detect clusters in the data that define the clustering boundaries capable of segregating subjects into groups matching health status categories. For example, k-means clustering, hierarchical clustering, neural networks or any other clustering method may be utilized in one or more embodiments of the invention. Furthermore, an embodiment of the invention may conduct the steps of FIG. 1 using a plurality of methods of clustering the data to achieve the results of the invention. The final clustering method that may be retained in any particular embodiment of the invention may be the one that yields the highest success rate of the diagnosis.

Step 170 represents computing success scores of the method of segregating of subjects in the test group. If the success level of the segregation into health categories is not satisfactory (e.g., no statistical difference compared to a population drawn from a random process), the parameters for computing the index values are revised and the analysis is repeated at step 140. The process of searching for optimal parameters may be repeated until the result of classification of subjects reaches (or exceeds) an acceptable success rate. Otherwise, if no optimal parameters may be found, the result may indicate that the chosen set of biomarkers is unsuitable for segregating the subjects, based on the index method under consideration, into the proposed health status categories.

The search for optimal parameters may involve changing one or more boundary values for discretizing biomarker values, and/or the weight coefficients associated with each biomarker in computing the index value for each subject. The search method may be manual i.e. an expert practitioner may set the initial parameters and adjust them, through multiple iterations of computation, while considering the outcome of the success rate of classification of subjects into health status categories. Implementations of the invention may also use numerical methods for automatic search to optimize parameters. Such methods comprise brute force search, where a large number of values of parameters and combinations thereof are tested. The numerical methods for determining optimal values may use gradient descent search, random walk search or any other mathematical method for searching for optimal parameters in order to achieve the goal of maximizing the success rate of the classification of subjects into correct corresponding health status categories.

Computer programs for conducting a search, in accordance with an implementation of the invention, require ordinary skills in the art of computer programming. Moreover, existing computer programs may be adapted (through a programming scripting language) to carry out a search process in an implementation of the invention. Any available computer program may be used, including, for example, the following computer programs identified by their respective registered trademark as follows: Mathematica™, Matlab™′ Medcalc™.

Step 180 represent the final step of determining the final parameters (or range thereof) that may be used in a diagnosis of the target disease(s). The optimal parameters include the coefficient associated with each biomarker, the number of ranges and the boundary values that define the ranges for each biomarker. Step 180 also includes determining the index range boundaries that define the categories as defined by the health status of subjects. The latter parameters may be used in systems for diagnosing whether a subject is a carrier of the a disease, as will detailed below in the method of use.

The invention provides a means for facilitating the display and read out of the results by defining the boundaries between ranges as discrete values for ease of use. For example, a scale comprising two health statuses, such as “disease present” and “disease not present”, may be defined has having a discrete boundary, such as one “1”, where the scale range lower than “1” may be mapped to “disease not present” status, while the scale range greater than “1” is mapped to “disease present” status.

Defining range boundaries as discrete values may be carried out during the search for the optimal parameters (as described above). The discrete range boundary values may also be provided computationally (e.g., using multipliers and offsets) subsequent to determining the optimal parameters.

An embodiment of the invention is specifically implemented to point out subjects with a high probability of being affected by a vector-borne disease. The latter implementation involves using the biomarkers: thymidine kinase 1 (TK1) and c-reactive protein (CRP). The following details show of how a practitioner is enabled to compute the VB index for a particular human subject and rule in (or rule out) that the subject has a high probability of being affected by a vector-borne disease. The details also demonstrate how the method according to the invention allows a practitioner to differentiate whether the subject may be affected by a vector-borne disease as opposed to other ailments (e.g., lymphosarcoma or irritable bowel disease).

In the following method, the level of TK1 is measured using its enzymatic activity and expressed in units per liter of blood (U/L), whereas the amount of CRP is measured by its mass and expressed in milligrams per liter of blood (mg/L). Both biomarkers can be determined in a sample of blood from human subjects.

### Method 1.

In an embodiment of the invention, an index for screening for VBD is computed using a discretization scheme as shown in Table 2 below.

TABLE 2

| TK1 U/L | CRP mg/L | Assigned |
|---|---|---|
| <=7.4 | >=4.1 | 0 |
| >=7.5 and <=12.2 | >=1.4 and <=4.0 | 1 |
| >=12.3 | <=1.3 | 2 |

The index is then computed using the following formula:

VBI1=(3.2*dTK1)+(1.7*dCRP)

Where VBI1 stands for the computed index in accordance with method 1; “dTK1” is the discrete value of TK1 and “dCRP” is the discrete value for the amount of CRP according to the discretization mapping provided in Table 2.

### Method 2

In another embodiment of the invention, the index may be computed using TK1 and CPR as shown in Table 3, as follows:

TABLE 3

| TK1 U/L | | CRP mg/L | Index Value (VBI2) |
|---|---|---|---|
| <12.3 | OR | >4.0 | 0 |
| >=12.3 | AND | <=4.0 | 1 |

In the latter case a subject is declared as affected by VBD only if TK1 level is greater than or equal to 12.3 U/L and CRP is less than or equal to 4.0 mg/L in the blood, otherwise the index is assigned the value “0” for ease of data comparison with other subjects and/or data sets.

### Method 3

In another embodiment of the invention, TK1 alone is considered as a biomarker for screening. A cutoff value of 7.5 U/L yields a significant result in detecting VBD as shown below in the several tables and the drawings.

Although other affections such as LSA or IBD may in fact be the cause of an elevation in TK1, these are far less common than VDB in the general population. As such, TK1 alone may be considered as a rapid test for determining the likelihood that a subject is affected by VBD. Once the TK1 screen test reveals a positive case, a second screen test with CRP may then be carried out to distinguish whether the underlying affection is caused by VBD or a different affection.

### Testing for VBD Versus Other Affections

As previously mentioned, TK1 and/or CRP may change under one or several health affections. To further validate the methods of the invention, several studies were carried out to compare several categories of affections and demonstrate the efficacy of the invention (especially in Method 1) to screen for VBD-affected subjects versus other diseases.

Subjects were classified in groups (categories) according to the health status as determined by a thorough diagnosis. Thus, a group of subjects affected by Lymphosarcoma (LSA), a group affected by Irritable Bowel Disease (IBD) and a normal (unaffected) group were pooled along with a group affected by Lyme disease. The analyses and especially embodiments of the invention are shown to specifically screen for those subjects specifically affected by VBD.

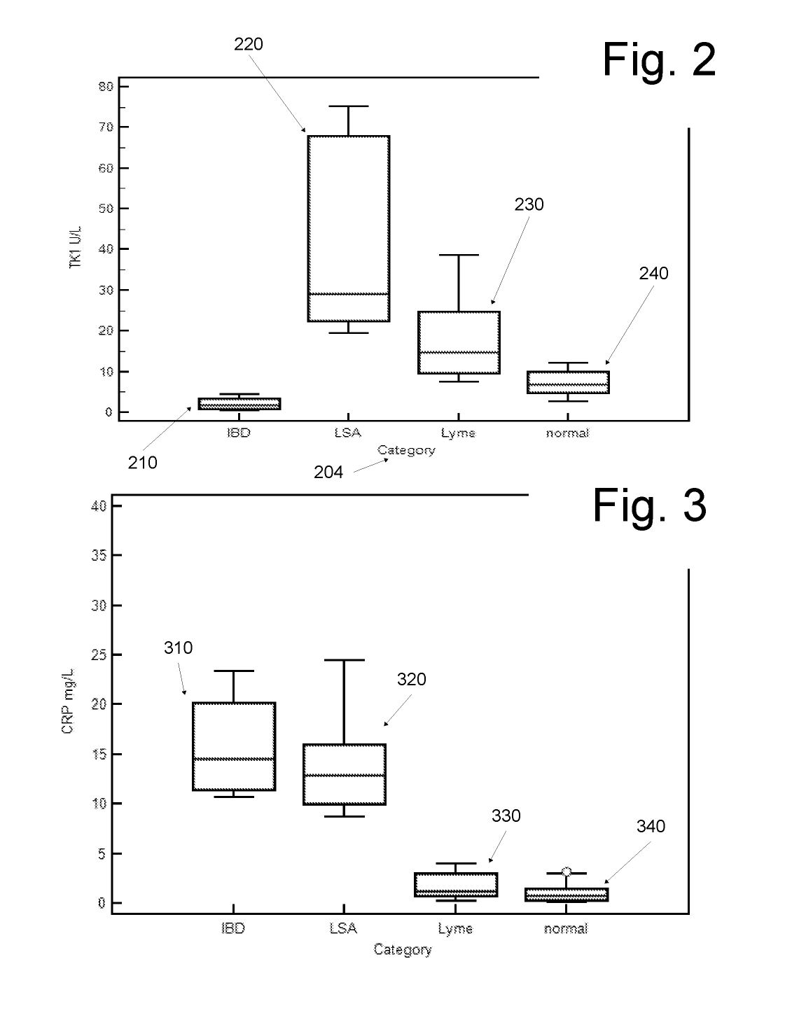

FIG. 2 is a bar chart representing statistics of thymidine kinase 1 activity level in several groups affected, respectively, by irritable bowel disease, lymphosarcoma and Lyme disease, and a normal (unaffected) group. TK1 level was measure in a blood sample of each subject and the statistical aggregates computed for each category (204). The chart of FIG. 2 shows that whereas TK1 levels in the group affected by IBD (210) is close to normal (240), the levels of TK1 in LSA group (220) and Lyme (230) are elevated compared with the normal (240).

FIG. 3 is a bar chart representing statistics of c-reactive protein concentration for several groups affected, respectively, by irritable bowel disease, lymphosarcoma and Lyme disease, and a normal (unaffected) group. The latter chart shows that CRP is elevated in the IBD (310) and LSA (320) groups compared to normal (340), whereas CRP in the group affected by Lyme disease (330) remains close to normal (340).

Receiver Operating Characteristic (ROC) analysis was carried out using TK1 alone, CRP alone, VBI1 and VBI2 as a variable to screen for VBD. The analysis compares the success rate of each variable.

The ROC analysis was carried on a group of fifty one (51) human subjects, twenty two (22) of whom are known to be affected by VBD. The results of the ROC analysis are summarized Table 4 below. In the following tables, the standard error (SE) is computed according to Elizabeth R. DeLong, David M. DeLong and Daniel L. Clarke-Pearson Biometrics Vol. 44, No. 3 (September, 1988), pp. 837-845. The confidence interval (CI) is taken as AUC±1.96 SE.

TABLE 4

| Variable | AUC | SE | 95% CI |
|---|---|---|---|
| TK1 | 0.788 | 0.0657 | 0.660 to 0.917 |
| CRP | 0.534 | 0.0823 | 0.373 to 0.696 |
| VBI1 | 0.947 | 0.0241 | 0.899 to 0.994 |
| VBI2 | 0.818 | 0.0525 | 0.715 to 0.921 |

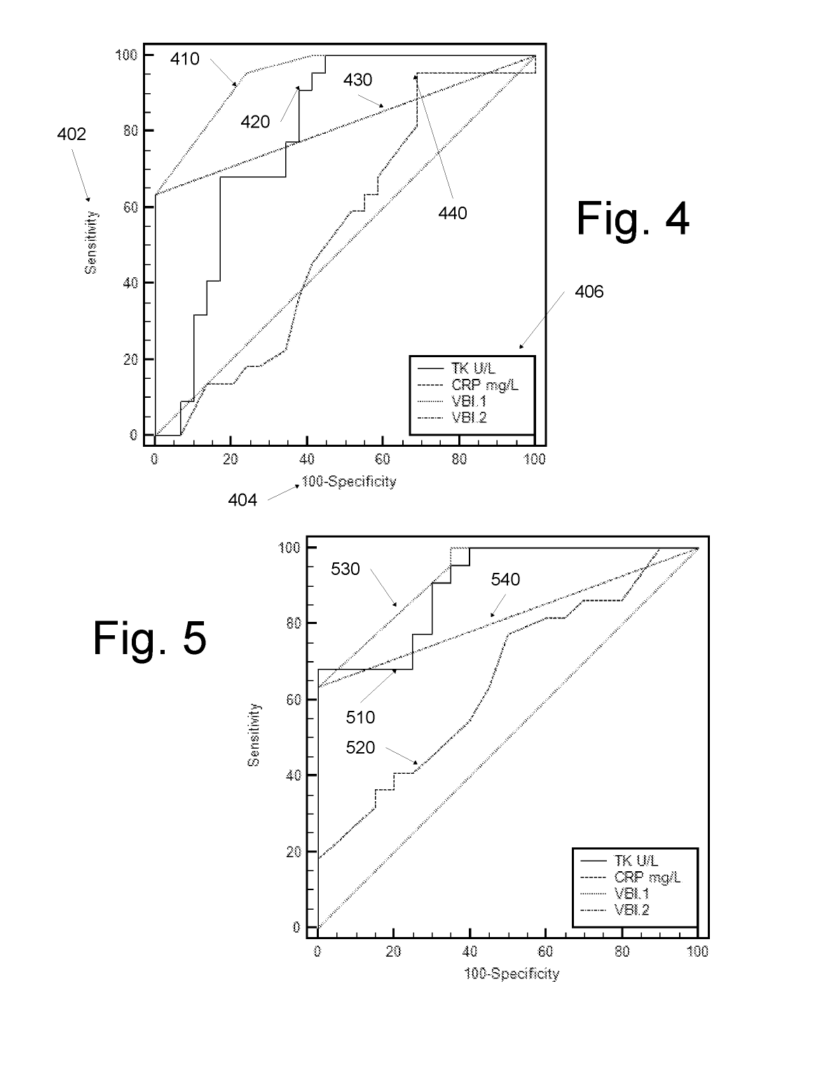

FIG. 4 plots the results of the Receiver Operating Characteristic (ROC) analysis carried out using TK1, CRP, VBI1 and VBI2. FIG. 4 plots a curve for each variable representing the sensitivity (402) as a function of 100 minus specificity (404). The area under the curve (AUC) is indicative of the propensity of the variable at screening for VBD. A higher AUC is indicative that the variable is more suited to determine true positives with minimum errors (false positives). FIG. 4 and Table 4 show that the biggest AUC is obtained using method 1 (VBI1) (410). Table 4 shows AUC of 0.947 (94.7%) for VBI1.

The AUC obtained with VBI2 (430) is smaller than that of VBI1, but remains satisfactory, at 0.818 (81.8%), when determining true positives.

Using TK1 alone yields an AUC (420) of 0.788 (78.8%); and CRP alone (440) yields an AUC of 0.534 (53.4%). Thus CRP poorly tests for the presence of VBD.

Tables 5a through 5f show the results of pairwise comparison of ROC curves statistics compared for TK, CRP, VB1 and VB2 categories.

TABLE 5a

| | | |
|---|---|---|
| TK vs CRP | Difference between areas | 0.254 |
| | Standard Error | 0.124 |
| | 95% Confidence Interval | 0.0103 to 0.498 |
| | z statistic | 2.043 |
| | Significance level | P = 0.0410 |

TABLE 5b

| | | |
|---|---|---|
| TK vs VBI1 | Difference between areas | 0.158 |
| | Standard Error | 0.0649 |
| | 95% Confidence Interval | 0.0311 to 0.285 |
| | z statistic | 2.440 |
| | Significance level | P = 0.0147 |

TABLE 5c

| | | |
|---|---|---|
| TK vs VBI.2 | Difference between areas | 0.0298 |
| | Standard Error | 0.0690 |
| | 95% Confidence Interval | −0.105 to 0.165 |
| | z statistic | 0.432 |
| | Significance level | P = 0.6660 |

TABLE 5d

| | | |
|---|---|---|
| CRP vs VBI1 | Difference between areas | 0.412 |
| | Standard Error | 0.0773 |
| | 95% Confidence Interval | 0.261 to 0.564 |
| | z statistic | 5.330 |
| | Significance level | P < 0.0001 |

TABLE 5e

| | | |
|---|---|---|
| CRP vs VBI.2 | Difference between areas | 0.284 |
| | Standard Error | 0.102 |
| | 95% Confidence Interval | 0.0832 to 0.484 |
| | z statistic | 2.773 |
| | Significance level | P = 0.0056 |

TABLE 5f

| | | |
|---|---|---|
| VBI1 vs VBI2 | Difference between areas | 0.129 |
| | Standard Error^{a} | 0.0415 |
| | 95% Confidence Interval | 0.0472 to 0.210 |
| | z statistic | 3.099 |
| | Significance level | P = 0.0019 |

Further analysis has been carried out to further demonstrate that the methods according to the invention are capable of determining those subjects who are likely affected by VBD. Whereas the comparative analysis above was carried out within the subjects of all the selected categories (i.e., IBD, LSA, Lyme and Normal), further analysis has been been carried out without the LSA and IBD groups. The analysis was carried out using 42 subjects, 22 of which were identified as being affected by VBD. Table 6 shows the results of the ROC analysis with a sample of subjects that excluded LSA and IBD subjects.

TABLE 6

| Variable | AUC | SE | 95% CI |
|---|---|---|---|
| TK | 0.902 | 0.0448 | 0.815 to 0.990 |
| CRP | 0.655 | 0.0852 | 0.488 to 0.822 |
| VBI1 | 0.928 | 0.0314 | 0.867 to 0.990 |
| VBI2 | 0.818 | 0.0525 | 0.715 to 0.921 |

FIG. 5 plots the results of the Receiver Operating Characteristic (ROC) analysis carried out with TK1 data alone, CRP data alone, VBI1 and VBI2 in a sample of subjects that excludes those subjects with Lymphosarcoma and Irritable Bowel Disease. FIG. 5 plots a curve for each variable representing the sensitivity as a function of 100 minus specificity. FIG. 5 shows that the biggest AUC is obtained using VB1 method (530). Table 6 shows AUC of 0.928 (92.8%) for VBI1.

The AUC obtained with VBI2 (540) is smaller than that of VBI1, at 0.818 (81.8%), when determining true positives.

Using TK1 alone yields an AUC (510) of 0.902 (90.2%). It is noteworthy that the AUC of the ROC when using TK1 alone has increased for the group which excludes LSA and IBD. The latter result is not surprising as LSA subjects in particular show an increased level of TK1, and thus by excluding the LSA group from the data there is less interference between the LSA group and the Lyme group. Thus, using TK1 alone as a biomarker allows for screening for VBD. Therefore, in situations where a subject have already been pre-screened and ruled out for LSA, for example, an embodiment of the invention may utilize the screening with TK1 alone.

CRP alone (520) yields an AUC of 0.655 (65.5%), which makes CRP alone a poor test for the presence of VBD.

Tables 7a through 7f show the results of pairwise comparison of ROC curves statistics compared for TK, CRP, VB1 and VB2 categories in a sample of subject that excludes those subjects identified as being affected by LSA or IBD.

TABLE 7a

| | | |
|---|---|---|
| TK vs CRP | Difference between areas | 0.248 |
| | Standard Error^{a} | 0.0928 |
| | 95% Confidence Interval | 0.0659 to 0.430 |
| | z statistic | 2.670 |
| | Significance level | P = 0.0076 |

TABLE 7b

| | | |
|---|---|---|
| TK vs VBI.1 | Difference between areas | 0.0261 |
| | Standard Error^{a} | 0.0317 |
| | 95% Confidence Interval | −0.0359 to 0.0882 |
| | z statistic | 0.825 |
| | Significance level | P = 0.4091 |

TABLE 7c

| | | |
|---|---|---|
| TK vs VBI.2 | Difference between areas | 0.0841 |
| | Standard Error^{a} | 0.0424 |
| | 95% Confidence Interval | 0.000991 to 0.167 |
| | z statistic | 1.983 |
| | Significance level | P = 0.0473 |

TABLE 7d

| | | |
|---|---|---|
| CRP vs VBI.1 | Difference between areas | 0.274 |
| | Standard Error^{a} | 0.0951 |
| | 95% Confidence Interval | 0.0874 to 0.460 |
| | z statistic | 2.879 |
| | Significance level | P = 0.0040 |

TABLE 7e

| | | |
|---|---|---|
| CRP vs VBI.2 | Difference between areas | 0.164 |
| | Standard Error^{a} | 0.0897 |
| | 95% Confidence Interval | −0.0121 to 0.339 |
| | z statistic | 1.825 |
| | Significance level | P = 0.0680 |

TABLE 7f

| | | |
|---|---|---|
| VBI.1 vs VBI.2 | Difference between areas | 0.110 |
| | Standard Error^{a} | 0.0396 |
| | 95% Confidence Interval | 0.0325 to 0.188 |
| | z statistic | 2.781 |
| | Significance level | P = 0.0054 |

FIG. 6 plots the computed values for sensitivity and the specificity as a function of TK cut-off value for a sample of subjects comprising IBD, LSA, Lyme and Normal subjects. In FIG. 6, Sensitivity (610) decreases as the cut-off value of TK is increased while the specificity (620) increases. The TK1 values are given above in Table 4 and FIG. 4 (plot 420). In addition, the computed Youden index J is 0.5517 for the associated criterion of >7.4 U/L, which yields a Sensitivity 100% and Specificity of 55.17%.

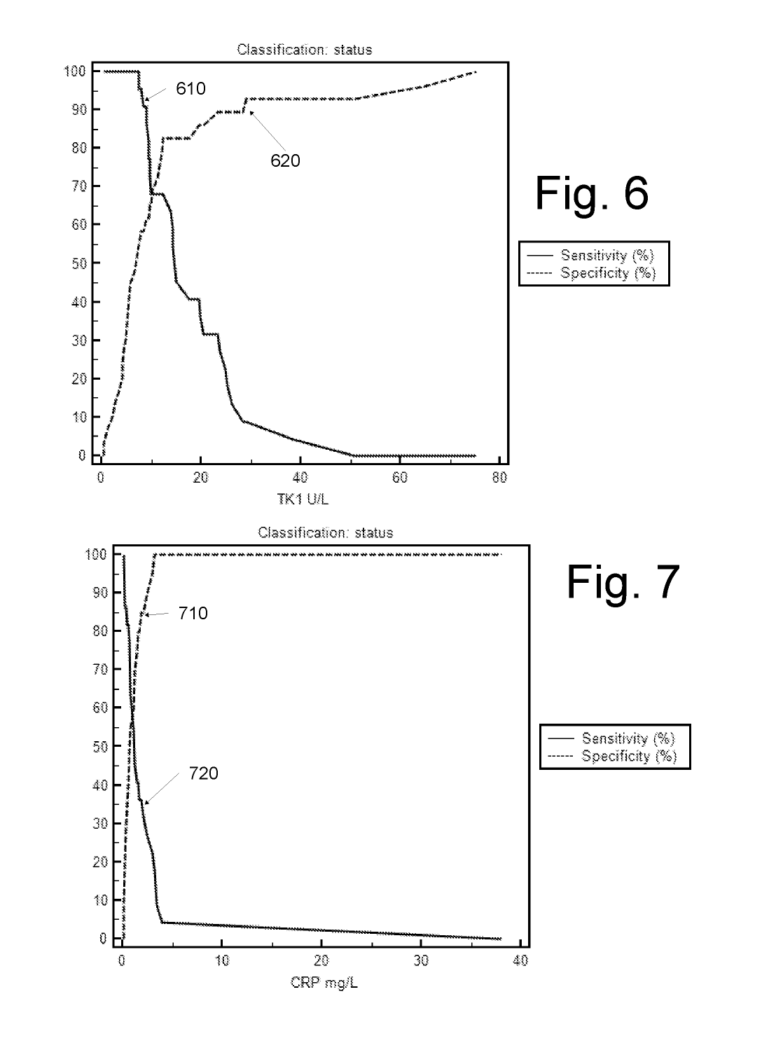

FIG. 7 plots the computed values for sensitivity and the specificity as a function of CRP cut-off values for a sample of subjects comprising IBD, LSA, Lyme and Normal subjects. In FIG. 7, Sensitivity (720) decreases as the cut-off value of CRP is increased while the specificity (710) increases. The CRP values are given above in Table 4 and FIG. 4 (plot 440). In addition, the computed Youden index J is 0.2649 for the associated criterion of ≦4 mg/L, which yields a Sensitivity 95.45% and Specificity of 31.03%.

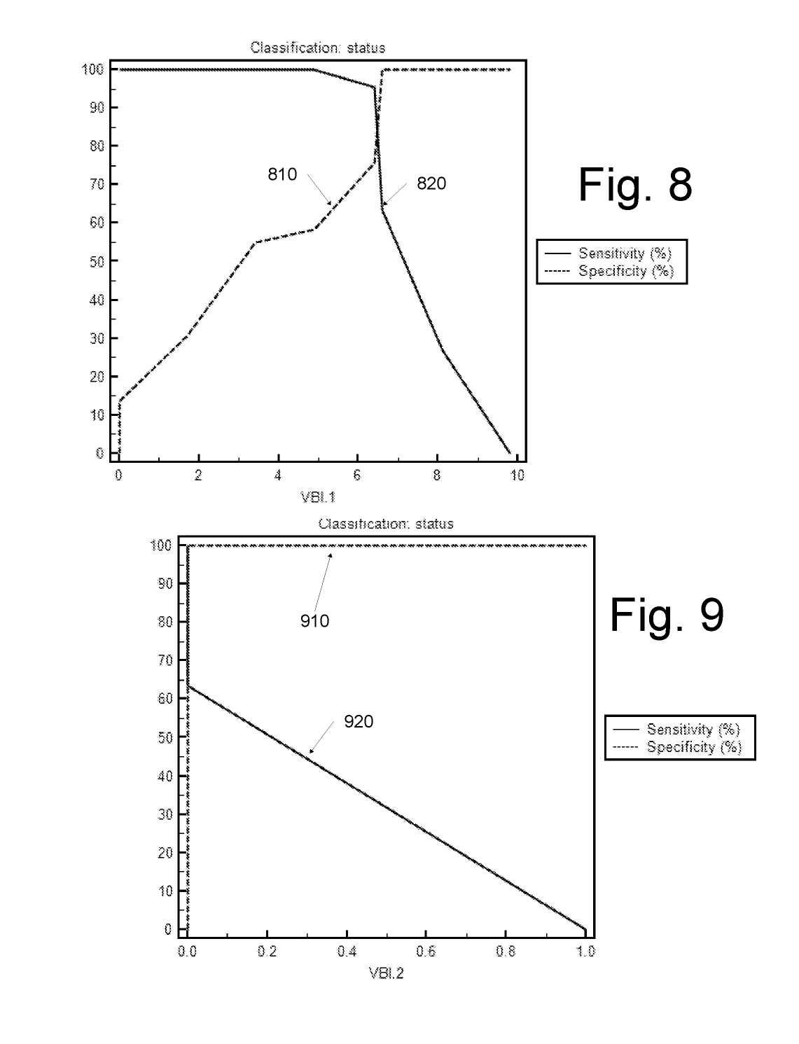

FIG. 8 plots the computed values for sensitivity and the specificity as a function of cut-off values of the vector-borne index according to method 1 (VBI1) for a sample of subjects comprising IBD, LSA, Lyme and Normal subjects. In FIG. 8, Sensitivity (820) decreases as the cut-off value of VBI1 is increased while the specificity (810) increases. The VBI1 AUC results are given above in Table 4 and FIG. 4 (plot 410). In addition, the computed Youden index J is 0.7132 for the associated criterion of >6.4, which yields a Sensitivity 95.45% and Specificity of 75.86%.

Table 8, below, shows the details of the results of the analysis plotted in FIG. 8 for the cut-off values encountered for the sample of subjects in the analysis.

TABLE 8

| Criterion | Sensitivity | 95% CI | Specificity | 95% CI | +LR | 95% CI | −LR | 95% CI |
|---|---|---|---|---|---|---|---|---|
| 0 | 100.00 | 84.6-100.0 | 0.00 | 0.0-11.9 | 1.00 | 1.0-1.0 | | |
| >4.9 | 100.00 | 84.6-100.0 | 58.62 | 38.9-76.5 | 2.42 | 1.6-3.7 | 0.00 | |
| >6.4 | 95.45 | 77.2-99.9 | 75.86 | 56.5-89.7 | 3.95 | 2.1-7.6 | 0.060 | 0.009-0.4 |
| >6.6 | 63.64 | 40.7-82.8 | 100.00 | 88.1-100.0 | | | 0.36 | 0.2-0.6 |
| >9.8 | 0.00 | 0.0-15.4 | 100.00 | 88.1-100.0 | | | 1.00 | 1.0-1.0 |

FIG. 9 plots the computed values for sensitivity and the specificity as a function of cut-off values of the vector-borne index according to method 2 (VBI2) for a sample of subjects comprising IBD, LSA, Lyme and Normal subjects. In FIG. 9, Sensitivity (920) decreases as the cut-off value of VBI2 is increased while the specificity (910) increases. VBI2 by design takes only one of two values “0” or “1”. The VBI2 AUC results are given above in Table 4 and FIG. 4 (plot 430). In addition, the computed Youden index J is 0.6364 for the associated criterion of >0, which yields a Sensitivity 63.64% and Specificity of 100%.

Further analyses have been carried out using a sample of subjects that excludes LSA and IBD affected subjects. The results are summarized in Table 9 below.

TABLE 9

| Var- | | | | Youden | | | |
|---|---|---|---|---|---|---|---|
| iable | AUC | SE | 95% CI | Index | criterion | Sensit. | Specif. |
| TK1 | 0.902 | 0.0448 | 0.815 to | .6818 | >12.2 | 68.18% | 100% |
| | | | 0.99 | | | | |
| CRP | 0.655 | 0.0852 | 0.488 to | 0.2727 | >0.7 | 77.27% | 50% |
| | | | 0.822 | | | | |
| VBI1 | 0.928 | 0.0314 | 0.867 to | 0.650 | >4.9 | 100% | 65% |
| | | | 0.990 | | | | |
| VBI2 | 0.818 | 0.0525 | 0.715 to | 0.6364 | >0 | 63.64% | 100% |
| | | | 0.921 | | | | |

Thus, a method and apparatus for enabling a practitioner to screen subjects as having a high probability of being affected by a vector-borne disease.

What is claimed is:

1. A method for screening human subjects for vector-borne diseases comprising the steps of:
obtaining a serum portion of a blood sample from a human subject;
obtaining an activity level of thymidine kinase in said serum portion;
obtaining a concentration of c-reactive protein in said serum portion; and
determining that said human subject has a high probability of being affected by a vector-borne disease if said activity level of thymidine kinase is greater than or equal to 7.5 U/L and less than or equal to 12.2 U/L and said concentration of c-reactive protein is greater than or equal to 1.4 mg/L and less that or equal to 4 mg/L.

2. The method of claim 1, further comprising determining that said human subject has a high probability of being affected by a vector-borne disease if said activity level of thymidine kinase is greater than 12.3 U/L, and obtaining a first multiplication product by multiplying said digitized thymidine kinase by 3.2 and said concentration of c-reactive protein is less than or equal to 1.3 mg/L, and obtaining a second multiplication product by multiplying said digitized c-reactive protein value by 1.7.

3. The method of claim 2, further comprising:
obtaining a digitized thymidine kinase value, wherein said digitized thymidine kinase value is assigned a value of zero (0) if said activity level of thymidine kinase is less than or equal to 7.4 U/L, said digitized thymidine kinase value is assigned a value of one (1) if said activity level of thymidine kinase is greater than or equal to 7.5 U/L and less than or equal to 12.2 U/L and said digitized thymidine kinase value is assigned a value of two (2) if said activity level of thymidine kinase is greater than 12.3 U/L, and obtaining a first multiplication product by multiplying said digitized thymidine kinase by 3.2;
obtaining a concentration of c-reactive protein in said serum portion, and obtaining digitized c-reactive protein value, wherein said digitized c-reactive protein value is assigned a value of zero “0” if said concentration of c-reactive protein is greater than or equal to 4.1 mg/L, said digitized c-reactive protein value is assigned a value of one “1” if said concentration of c-reactive protein is greater than or equal to 1.4 mg/L and less that or equal to 4 mg/L, and said digitized c-reactive protein value is assigned a value of two “2” if said concentration of c-reactive protein is less than or equal to 1.3 mg/L, and obtaining a second multiplication product by multiplying said digitized c-reactive protein value by 1.7;
computing an index by summing said first multiplication product and said second multiplication product; and
determining that said human subject has a high probability of being affected by a vector-borne disease if said index is greater than or equal to one “1”.

4. A system for screening human subjects for vector-borne diseases comprising:
means for obtaining a serum portion of a blood sample from a human subject;
means for obtaining an activity level of thymidine kinase in said serum portion;
means for obtaining a concentration of c-reactive protein in said serum portion; and
means for determining that said human subject has a high probability of being affected by a vector-borne disease if said activity level of thymidine kinase is greater than or equal to 7.5 U/L and less than or equal to 12.2 U/L and said concentration of c-reactive protein is greater than or equal to 1.4 mg/L and less that or equal to 4 mg/L.

5. The system of claim 4 further comprising means for determining that said human subject has a high probability of being affected by a vector-borne disease if said activity level of thymidine kinase is greater than 12.3 U/L, and obtaining a first multiplication product by multiplying said digitized thymidine kinase by 3.2 and said concentration of c-reactive protein is less than or equal to 1.3 mg/L, and obtaining a second multiplication product by multiplying said digitized c-reactive protein value by 1.7.

6. The system for claim 5, further comprising:
means for obtaining a digitized thymidine kinase value, wherein said digitized thymidine kinase value is assigned a value of zero (0) if said activity level of thymidine kinase is less than or equal to 7.4 U/L, said digitized thymidine kinase value is assigned a value of one (1) if said activity level of thymidine kinase is greater than or equal to 7.5 U/L and less than or equal to 12.2 U/L and said digitized thymidine kinase value is assigned a value of two (2) if said activity level of thymidine kinase is greater than 12.3 U/L, and obtaining a first multiplication product by multiplying said digitized thymidine kinase by 3.2;
means obtaining a concentration of c-reactive protein in said serum portion, and obtaining digitized c-reactive protein value, wherein said digitized c-reactive protein value is assigned a value of zero “0” if said concentration of c-reactive protein is greater than or equal to 4.1 mg/L, said digitized c-reactive protein value is assigned a value of one “1” if said concentration of c-reactive protein is greater than or equal to 1.4 mg/L and less that or equal to 4 mg/L, and said digitized c-reactive protein value is assigned a value of two “2” if said concentration of c-reactive protein is less than or equal to 1.3 mg/L, and obtaining a second multiplication product by multiplying said digitized c-reactive protein value by 1.7;
means for computing an index by summing said first multiplication product and said second multiplication product; and
means for determining that said human subject has a high probability of being affected by a vector-borne disease if said index is greater than or equal to one “1”.

7. A method for screening human subjects for vector-borne diseases comprising the steps of:
obtaining a serum portion of a blood sample from a human subject;
obtaining an activity level of thymidine kinase in said serum portion;
obtaining a concentration of c-reactive protein in said serum portion; and
determining that said human subject has a high probability of being affected by a vector-borne disease if said activity level of thymidine kinase is greater than 12.3 U/L and said concentration of c-reactive protein is less than 4 mg/L.

8. The method of claim 7, further comprising:
computing an index having a value of one “1” if said activity level of thymidine kinase is greater than 12.3 U/L and said concentration of c-reactive protein is less than 4 mg/L, said index having a value of “0” otherwise; and
determining that said human subject has a high probability of being affected by a vector-borne disease if said index is equal to one “1”.
